(19) United States
(12) Patent Application Publication
(10) Pub. No.: US 2010/0204478 A1
(43) Pub. Date: Aug. 12, 2010

(54) IMPROVED PROCESS FOR AMOPHOUS RABEPRAZOLE SODIUM

(75) Inventors: Bandi Parthasaradhi Reddy, Hyderabad (IN); Kura Rathnakar reddy, Hyderabad (IN); Rapolu Raji Reddy, Hyderabad (IN); Dasari Muralidhara Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (AT)

(21) Appl. No.: 11/917,142

(22) PCT Filed: May 25, 2007
(86) PCT No.: PCT/IN07/00208

§ 371 Date: Dec. 10, 2007

(51) Int. Cl. C07D 401/12

(52) U.S. Cl. 546/273.7

(57) ABSTRACT

The present invention provides an improved and efficient process for the preparation of highly pure amorphous rabeprazole sodium. Thus, for example, rabeprazole is dissolved in an alcoholic sodium hydroxide solution followed by carbon treatment, the resulting filtrate is distilled under vacuum at 50-52° C. followed by co-distillation with cyclohexane and the resulting residue is dissolved in anisole; the solution is added to cyclohexane under agitation and then the precipitated solid collected by filtration or centrifugation.

### FIELD OF THE INVENTION

The present invention relates to an improved and efficient process for preparation of highly pure amorphous rabeprazole sodium.

### BACKGROUND OF THE INVENTION

U.S. Pat. No. 5,045,552 disclosed pyridine-2-ylmethylsulfinyl-1H-benzimidazole derivatives, process for their preparation, pharmaceutical compositions in which they are present and the use thereof. These compounds are H⁺/K⁺ ATPase inhibitors used for treatment of diseases caused due to increased gastric acid secretion. An especially important compound among those disclosed is rabeprazole sodium, chemically 2-[[[4-(3-methoxypropoxy)-3-methyl-2-pyridinyl]methyl]sulfinyl]-1H-benzimidazole sodium salt, is an inhibitor of the gastric proton pump. It belongs to a class of antisecretory compounds that do not exhibit anticholinergic or histamine H2-receptor antagonist properties, but suppress gastric acid secretion by inhibiting the gastric H⁺/K⁺ ATPase at the secretory surface of the gastric parital cell. Rabeprazole blocks the final step of gastric acid secretion. Rabeprazole sodium is represented by the following structure:

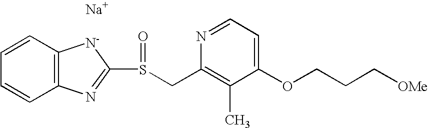

As per the process described and exemplified in the U.S. Pat. No. 5,045,552, rabeprazole sodium is prepared by oxidizing 2-[[4-(3-methoxypropoxy)-3-methylpyridine-2-yl]methylthio]-1H-benzimidazole with m-chloroperbenzoic acid to afford the rabeprazole base which is further converted to its sodium salt by using 0.1N aqueous solution of sodium hydroxide, followed by addition of ethanol. The water is removed by azeotropic distillation and the product is precipitated by using ether as solvent such as diethyl ether, tert-butyl methyl ether. The melting point of the disclosed rabeprazole sodium salt is 140-141° C.

The isolation process described in the U.S. Pat. No. 5,045,552 has numerous disadvantages such as large volume of solvents is required for azeotropic removal of water during which the product is exposed to high temperature and leads to certain impurities. Based on these drawbacks the isolation process finds to be unsuitable for preparation of amorphous rabeprazole sodium at commercial scale operations.

Japanese patent application JP 2001039975 indicates that the product obtained by example 33 of the U.S. Pat. No. 5,045,552 with a melting point of 140-141° C. corresponds to amorphous rabeprazole sodium. In this application, the X-ray powder diffraction pattern of the amorphous rabeprazole sodium is shown.

The PCT patent publication No. WO 03/101452 discloses a method for the preparation of rabeprazole sodium comprising dissolving rabeprazole base in aqueous sodium hydroxide and then subjecting to lyophilization.

U.S. Pat. No. 6,180,652 B1 (the '652 patent) describes acetone complex of rabeprazole sodium, process for its production and characterizes it by powder X-ray diffraction, infra-red spectroscopy and ¹H-NMR spectroscopy. The '652 patent further reports a process for preparation of amorphous rabeprazole sodium by lyophilizing (freeze-drying) an aqueous solution of rabeprazole sodium acetone complex.

However, lyophilization is a technique, which is not suitable for production at industrial scale because this process presents serious limitations on cost, time, equipment capability and environmental protection.

According to PCT patent publication No. WO 2004/085424A1, amorphous rabeprazole sodium is obtained by heating the rabeprazole sodium acetone complex at elevated temperature, preferably between 100 and 110° C. It is well known that exposing rabeprazole-type compounds to high temperatures increases the risk of decomposition to form impurities and as such, heat treatment of rabeprazole sodium acetone complex into amorphous rabeprazole sodium is not adequate for the production of a rabeprazole which is suitable for pharmaceutical use.

PCT patent publication No. WO 2007/023393 A2 reports a process for preparation of amorphous rabeprazole sodium, the said process comprises: i) contacting rabeprazole sodium acetone complex with a first solvent system which includes a hydrocarbon solvent or an ether solvent or an alcohol solvent or mixtures thereof; ii) filtering the solid from the solvent system used in step i) or distilling the solvent system used in step i) under reduced or atmospheric pressure, to thereby obtain a residue; iii) contacting the wet solid or the residue of step ii) with a second solvent system which includes a hydrocarbon solvent or an ether solvent; and iv) filtering to obtain a wet solid from the solvent system used in step iii) to obtain a wet solid.

The methods for preparation of amorphous rabeprazole sodium as described in the U.S. Pat. No. 6,180,652 B1, PCT patent publication No. WO 2004/085424A1 and PCT patent publication No. WO 2007/023393 A2 involves lengthy process i.e., proceeds via rabeprazole sodium acetone complex intermediate and also the yields obtained in these processes are very low.

U.S. Patent Application No. US2004/0180935A1 teaches a process for production of amorphous rabeprazole sodium by dissolving rabeprazole acid in a mixture of sodium hydroxide and methanol at 25-35° C., removing the solvent by evaporation and precipitating the product by adding petroleum ether.

PCT patent publication No. WO 2006/120701 A1 teaches a process for manufacture of amorphous rabeprazole sodium with mean particle diameter between 10 to 55 μm, the said process comprises, addition of rabeprazole to aqueous sodium hydroxide; addition of ethyl alcohol to the solution; distillation of solvents from the solution thus obtained till thick mass is obtained; addition of an organic solvent selected from ethyl acetate, dichloromethane, chloroform, butyl acetate, ethanol, isopropyl alcohol, methanol, tetrahydrofuran, to the residue to obtain a clear solution; addition of this clear solution to an anti-solvent includes diisopropyl ether, diethyl ether, methyl tert-butyl ether, under agitation and isolation of the product.

Since a solvent may play an important role in increasing the yield rate or in determination of physical properties of drug substance such as crystal form, purity, solubility, etc., even if such a solvent is known to be toxic, there may be many cases that the use thereof in the preparation of drug substance cannot be avoided in terms of risk benefits. In such cases, this guideline (ICH guidelines Q3C(R3)) decrees that a concentration of a residual solvent in drug substance should be not more than a specified value, which is toxicologically acceptable.

The methods for preparation of amorphous rabeprazole sodium as described in the patents, U.S. Patent Application No. US2004/0180935A1 and PCT patent publication No. WO 2006/120701 A1 suffers with residual solvent problem and thereby commercially not viable. These methods utilize the solvents like diisopropyl ether and petroleum ether as precipitating solvents. These solvents are difficult to remove completely by practical manufacturing techniques. According to the ICH guidelines Q3C(R3), there is no adequate toxicological data for the solvents like diisopropyl ether and petroleum ether on which to base a PDE was found.

However, a need still remains for an improved and commercially viable process of preparing pure amorphous rabeprazole sodium that would solve the aforesaid problems associated with processes described in the prior art, which will be suitable for larger-scale preparation, in terms of simplicity, chemical yield and purity of the product, and which would carry out with comparatively smaller volume of solvent.

It has been surprisingly found that the amorphous rabeprazole sodium can be obtained in high purity and in high yield when aromatic ether, preferably anisole is used as the solvent in relatively smaller amounts. The process is more economic in addition to being eco-friendly.

### DETAILED DESCRIPTION OF THE INVENTION

In accordance with the present invention, a process is provided for the preparation of highly pure amorphous rabeprazole sodium, which comprises:
a) dissolving rabeprazole in an alcoholic sodium hydroxide solution;
b) subjecting the solution obtained in step (a) to carbon treatment;
c) removing the alcoholic solvent from the filtrate obtained in step (b) under vacuum at a temperature ranging between 50-55° C. to obtain a residue;
d) dissolving the residue obtained in step (c) in an aromatic ether solvent, preferably anisole, to obtain a solution;
e) adding the solution obtained in step (d) to an anti-solvent under inert atmosphere; and
f) collecting amorphous rabeprazole sodium.

Rabeprazole used as starting material may be obtained by processes described in the art, for example by the process described in the U.S. Pat. No. 5,045,552.

The alcoholic sodium hydroxide solution used in step (a) is prepared by dissolving sodium hydroxide in an alcoholic solvent at an elevated temperature i.e., between 35° C. and about 80° C., preferably between about 60° C. and about 70° C.

The alcoholic solvent used in step (a) is selected from a group consisting of methanol, ethanol, n-propanol and isopropanol. Preferable alcoholic solvent is isopropanol.

Rabeprazole in step (a) is dissolved in alcoholic sodium hydroxide solution preferably at an ambient temperature i.e., between about 25° C. and about 40° C., and more preferably between 30° C. and 40° C.

Preferable aromatic ether solvent used in step (d) is anisole.

The residue obtained in step (c) is dissolved in anisole preferably at a temperature between about 20° C. and about 50° C., and more preferably between 30° C. and 40° C.

The anti-solvent used in step (e) is a hydrocarbon solvent selected from the group consisting of n-pentane, n-hexane, n-heptane and cyclohexane. Preferable anti-solvent is cyclohexane.

The solution obtained in step (d) is added to cyclohexane preferably at a temperature between about 20° C. and about 40° C., and more preferably between 30° C. and 40° C.

The solvent in step (d) is used in an amount of 2 to 4 ml and the anti-solvent in step (e) is used in an amount of 15 to 17 ml per gram of rabeprazole.

The solution in step (e) is preferably stirred at least for about 30 minutes, more preferably stirred at least for about 1 hour and still more preferably stirred for about 1 hour to 2 hours.

The amorphous rabeprazole sodium obtained in step (f) is collected by filtration or centrifugation.

The process ensures the high purity. The purity (by ‘High Performance Liquid Chromatography’, herein after referred to as HPLC) of the product obtained according to the present invention is preferably about above 98%, more preferably about above 99% and still more preferably about above 99.5%.

### BRIEF DESCRIPTION OF THE DRAWING

FIG. 1 shows the X-ray diffraction pattern of amorphous rabeprazole sodium.

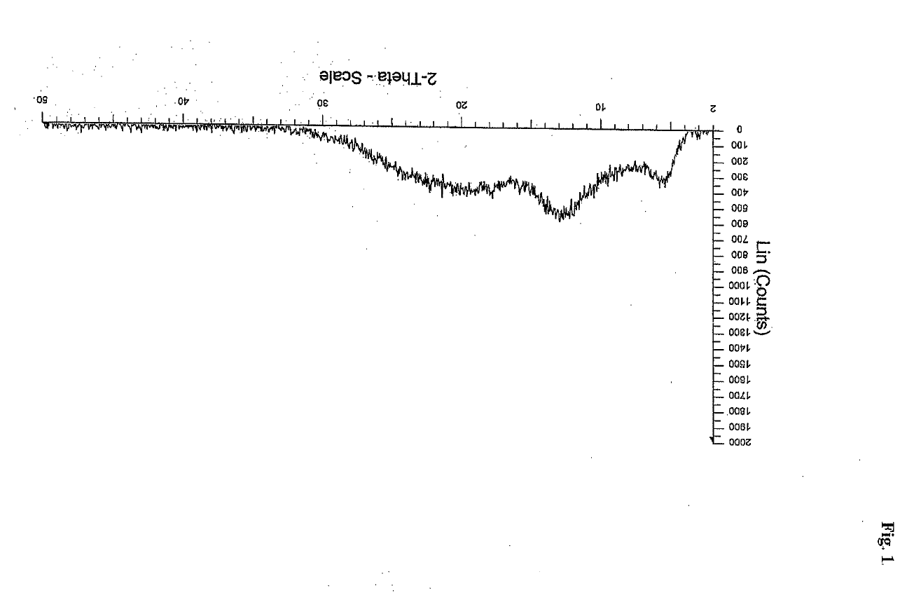

X-Ray powder diffraction spectrum was measured on a Bruker axs D8 advance x-ray powder diffractometer having a Copper-Kα radiation. Approximately 1 gm of sample was gently flattened on a sample holder and scanned from 2 to 50 degrees two-theta, at 0.03 degrees two-theta per step and a step time of 0.5 seconds. The sample was simply placed on the sample holder. The sample was rotated at 30 rpm at a voltage 40 KV and current 35 mA.

The invention will now be further described by the following example, which is illustrative rather than limiting.

### Example

Isopropyl alcohol (300 ml) is added to sodium hydroxide (2.5 gm) under stirring, the contents are heated to 60-65° C. until to form a clear solution and then cooled to 30° C. To the solution added rabeprazole (25 gm) for 20 minutes under nitrogen atmosphere, stirred for 30 minutes and then activated carbon (2 gm) is added under stirring. Filtered the mass on hiflow, washed with isopropyl alcohol (50 ml) and the resulting filtrate is distilled under vacuum at 50-52° C. The residue is co-distilled with cyclohexane (150 ml) and then dissolved in anisole (75 ml). The resulting mass is slowly added to cyclohexane (400 ml) under nitrogen atmosphere at 30-35° C. for 45 to 50 minutes and then stirred for 1 hour at 30° C. Filtered the mass, the separated solid is washed with cyclohexane (25 ml) and then dried the material at 60-65° C. for 5 hours to yield 22 gm of amorphous rabeprazole sodium (HPLC purity: 99.9%).

We claim:

1. A process for preparation of highly pure amorphous rabeprazole sodium, which comprises:
a) dissolving rabeprazole in an alcoholic sodium hydroxide solution;
b) subjecting the solution obtained in step (a) to carbon treatment;
c) removing the alcoholic solvent from the filtrate obtained in step (b) under vacuum at a temperature ranging between about 50-55° C. to obtain a residue;
d) dissolving the residue obtained in step (c) in an aromatic ether solvent to obtain a solution;
e) adding the solution obtained in step (d) to an anti-solvent under inert atmosphere; and
f) collecting amorphous rabeprazole sodium.

2. The process as claimed in claim 1, wherein the aromatic ether solvent is anisole.

3. The process as claimed in claim 1, wherein the residue obtained in step (c) is dissolved in anisole at a temperature between about 20° C. and about 50° C.

4. The process as claimed in claim 3, wherein the residue is dissolved in anisole at a temperature between about 30° C. and 40° C.

5. The process as claimed in claim 1, wherein the anti-solvent used in step (e) is a hydrocarbon solvent selected from the group consisting of n-pentane, n-hexane, n-heptane and cyclohexane.

6. The process as claimed in claim 5, wherein the anti-solvent is cyclohexane.

7. The process as claimed in claim 1, wherein the solution obtained in step (d) is added to cyclohexane at a temperature between about 20° C. and about 40° C.

8. The process as claimed in claim 7, wherein the solution is added to cyclohexane at a temperature between about 30° C. and 40° C.
